(19) United States
(12) Patent Application Publication
(10) Pub. No.: US 2006/0282125 A1
(43) Pub. Date: Dec. 14, 2006

(54) Evaluation of cardiac function using only telemetered left ventricular pressure during axial flow LVAD support in sheep: comparison to preload recruitable stroke work

(76) Inventor: Patrick I. McConnell, Salt Lake City, UT (US)

Correspondence Address: ROETZEL & ANDRESS, 1375 EAST 9TH STREET, CLEVELAND, OH, 44114, US

(21) Appl. No.: 11/150,855

(22) Filed: Jun. 9, 2005

(51) Int. Cl. A61N 1/00

(52) U.S. Cl. 607/6

(57) ABSTRACT

Introduction: Emerging technology will soon allow for the chronic assessment of telemetered left ventricular (LV) pressure in patients supported with LVADs; however, specific methods have not been developed for the use of LV pressure during LVAD support. Methods: Adult Suffolk sheep (N=6, 78±3 Kg) underwent placement of axial-flow LVAD. Each animal was instrumented with a telemetered LV pressure manometer, an outflow graft transit time flow probe, and endocardial LV long and short axis piezoelectric crystals to derive LV volume and stroke work (SW). In unsedated sheep, LV load was varied by increasing LVAD speed from 6,000 to 10,880±120 rpm (“run”). During ‘run’, the PRSW (slope: M_{W}), and simultaneously, the relationship of LV triple product (TP) to LV end-diastolic pressure (TP/EDP, slope: M_{TP}) were determined by least squares regression analyses before and after β1-blockade (esmolol 5 mg/kg/min). Comparisons were made using One-way ANOVA and multiple linear regression analysis. Results: VAD support, 4.5±0.31 L/min, was maintained for >72 hrs prior to study. When VAD speed was increased from 6,000 rpm (‘run’), the LVAD flow (Q_{V}) increased (1.2±0.25 to 5.8±0.71 L/min) while the LV SW (3061±747 to 1556±410 mmHg*mL), LV TP (3127±397 to 1019±335×10⁵) and LV EDP (18.2±1.2 to 9.7±1.8 mm Hg) decreased (P<0.01). The relationships of TP-EDP and the PRSW established during LVAD ‘run’ were sensitive and reduced by esmolol administration (M_{TP}: 158±23.8 to 71±15.1; P<0.001 and Mw: 117±15.8 to 72±9.4; P<0.001). Conclusions: Like the PRSW, the relationship of TP to EDP established during LVAD unloading ‘run’, was sensitive to changes in cardiac function after esmolol administration. In the future, LV pressure-derived parameters could improve the outcomes of patients supported with mechanical assist devices.

Recently, the Pilot Clinical Study Evaluating an Implantable Left Ventricular Pressure Monitoring System in Patients with a Left Ventricular Assist Device Intended for Cardiac Transplantation or “VALAD” trial began enrolling patients in Germany. In this trial, a telemetered left ventricular (LV) pressure manometer (LVP1000®: Transoma Medical, St Paul, Minn.) is placed transmyocardially to monitor LV pressures during left ventricular assist device (LVAD) support. This or similar technology could potentially improve the care of patients who have both a LV pressure monitor and an assist device. There recently has been growing enthusiasm, based on an increasing number of reports, for the concept that left ventricular volume unloading with an LVAD may permit the recovery of cardiac function and remodeling sufficient for device explant-so-called ‘Bridge to Recovery’. Unfortunately, the reality of this therapeutic curiosity is that very few patients, likely less than 5% of chronic heart failure patients supported with a LVAD, demonstrate enough cardiac function for LVAD removal. However, there is agreement within the scientific community that with better diagnostic capabilities to direct device operation or concomitant therapeutics, better success rates could be realized. Including the relevant recommendations by the Working Group on Recovery from Heart Failure with Circulatory Assist of the National Heart, Lung and Blood Institute [Reinlib and Abraham. J Card Fail 2003 9: 459-63] for the serial determination of anatomical structure and functional parameters aimed at the proper assessment of recovery, for markers and predictive factors (hemodynamics) of ‘recoverable’ hearts to be identified and for the design of mechanical assist devices and systems specifically for cardiac recovery.

Therefore, if less arduous methods existed for evaluating LV function during LVAD support, permitting greater frequency or even automated assessments, it is likely that improvements in device operation and ‘weaning’ strategies could be realized allowing for better ‘Bridge to Recovery’ therapy. A reduction in adverse events and morbidity associated with ‘Destination’ therapies could also potentially be realized with chronic LV hemodynamic monitoring—by allowing the operation of devices at more appropriate support levels, device wear and some potentially negative effects to the native heart (e.g. right heart failure, arrhythmias, etc.) as well as the patient (e.g. thromboembolic events) could be reduced or avoided. Unfortunately, to date, no relevant or specific methods exist for determining cardiac function from the LV pressure signal alone in patients supported with an axial flow LVAD.

Ferrari and colleagues recently reported on monitoring 2 patients' load-independent cardiac function using pressure volume (P-V) analysis derived from the offline analysis of catheter acquired LV pressure signals and echocardiographically-derived LV volumes at implant and explant of an axial flow LVAD. In these cases, the establishment of the end-systolic pressure volume relationship (ESPVR) was performed in a novel way by using the LVAD to acutely unload the LV—establishing the P-V relationship. Yet, theoretical and technical issues related with the interpretation of end-systolic elastance or the ESPVR during axial-flow unloading might limit the interpretation of end-systolic elastance under axial flow unloading conditions. For example, as the LVAD unloads the LV, the systemic circulation is supported limiting changes in mean arterial pressure and the LV end-systolic pressure despite large changes in LV volume. A situation has then been created where changes in the end-systolic pressure are not dependent on changes in LV volume—compromising any index reliant on the coupling of these particular factors (i.e. ESPVR).

In light of developing technology, we have propose herein a method to assess cardiac function during axial-flow LVAD support that would allow for a) the frequent and b) the repetitive assessment of LV function from c) a single hemodynamic source—telemetered LV pressure. We have studied whether the relationship of the LV triple product (TP: LVSP*dP/dtₘₐₓ*HR) to LVEDP, TP/EDP (slope: M_{TP}), could provide an index sensitive to changes in cardiac function like the preload-recruitable stroke work (PRSW). Thus, in unsedated sheep supported with an axial-flow LVAD, we have compared the TP/EDP to the PRSW, before and after beta-adrenergic blockade with esmolol.

Materials and Methods

Studies were approved by the Institutional Lab Animal Care and Use Committee (ILACUC) at The Ohio State University, and adhered to the statutes of the Animal Welfare Act and the guidelines of the Public Health Service.

LVAD Placement and Instrumentation

Adult sheep (N=6, 78±3 Kg) underwent placement of an axial-flow LVAD (Heart Mate II®, Thoratec Corp., Pleasanton, Calif.) through a left thoracotomy while avoiding cardiopulmonary bypass. The LVAD inflow cannula was positioned through the LV apex and the outflow graft (16 mm) was sewn to the descending thoracic aorta. The pump remained within the thorax and the transcutaneous power cable was tunneled to the animals' left flank.

Fluid-filled catheters (Tygon®) were secured with suture into the descending thoracic aorta and left atrial appendage. In four of six animals (N=4), two pairs of piezoelectric crystals (2 mm, Sonometrics Inc., New London, Ontario Canada) were surgically placed endocardially in the equatorial plane at the mid papillary level (short axis, SA), and anteriorly at the LV base and near the LV apex (long-axis, LA) for calculation of LV volumes. Telemetered manometers (TL13-D70-PCP, Data Sciences International, St. Paul, Minn.) were secured within the right ventricle (RV, N=5) and the LV chambers. An ultrasonic transit-time flow probe (16 mm, Transonic Inc., Ithaca, N.Y.) was placed around the LVAD outflow graft. All catheters and cables were exited from between the animals' scapula. Prior to performing studies, animals were allowed to recover typically for at least 1 week while the LVAD was operated continuously at approximately 9,000 rpm (partial support).

Data Acquisition

Aortic and left atrial fluid filled catheters were connected to calibrated Statham pressure transducers (Model: P23XL; Biggo-Spectramed, Ocknard, Calif.) and amplified (Gould, Valley, Ohio) for their respective pressures. The telemetered pressure waveforms were acquired via UA-10 receiver (DSI, St Paul, Minn.) and electronically calibrated while adjusting for atmospheric conditions and the accuracy of LV pressure was confirmed against calibrated aortic and left atrial pressure signals. Sonomicrometer signals were analyzed for cardiac-cycle dependent (end-diastolic and end-systolic) and waveform dependent (minimum, maximum, mean etc) parameters. The signals from the outflow graft flow probe were amplified and electronically calibrated before each experiment. All waveforms were collected (at 1 kHz) and analyzed by a 16-channel data acquisition and software system (IOX, version 1.7, EMKA Technologies, Falls Church Va.). Hemodynamic waveforms were analyzed (IOX) and averaged data (2 second) was output to tab delimited files and accessed using standard spreadsheet program (Excel, Microsoft Inc., Redmond, Wash.).

Calculated Parameters

Left ventricular volume was calculated in real-time from endocardial positioned sonomicrometers using the equation: (SA²*LA*π/6)*1000 (ml). Left ventricular triple product (TP) was calculated on a per beat basis within software (IOX) from the telemetered LV pressure signal using the following equation: LVSP*dP/dtₘₐₓ*HR—where LVSP was the LV systolic pressure, dPdtₘₐₓ was the maximal derivative of LV pressure and HR was the heart rate. The LV stroke work (SW) was also calculated (∫LVP*DLV volume) in real-time within software (IOX) on a per beat basis.

Study Design(s)

Baseline LVAD supported data (‘on support’) was collected from awake, unrestrained and standing animals while the LVAD support was continued up to 10,000 rpm. LV unloading with the axial flow LVAD was performed after the pump speed was reduced to 6,000 rpm and the animals' hemodynamics were allowed to stabilize for up to 2 minutes at this speed. Then the LVAD was programmed to “run” up to a point where the LVSP<MAP or approximately 11,000 rpm (100 rpm/second). In each case, the TP/EDP relationship and the PRSW were derived from the same ‘run’.

Responses to Esmolol

The responses of TP/EDP and PRSW to changes in inotropy were evaluated after β1-adrenergic blockade with esmolol hydrochloride (????). On the same day as the baseline ‘run’, animals were administered an intravenous bolus of esmolol (25 mg) followed by intravenous esmolol infusion (5 mg/kg/min). A ‘run’ was repeated after at least 1 minute of esmolol infusion.

Responses to Phenylephrine (PE)

The effect of increased afterload on the TP/EDP relationship was assessed in a single animal on three separate days. Prior to each study, autonomic blockade was produced with atropine (0.1 mg/kg i.v.) and metoprolol (5 mg i.v.) to prevent baroreflex activation during PE infusion. Phenylephrine was infused at 0.01, 0.1 and 0.25 mcg/kg/min with the goal to increase LV systolic pressure by approximately 10, 20 and 30 mm Hg, respectively.

Statistics

Data is expressed as the mean±SEM. Data was collected during a single experimental period or day; therefore, comparisons of hemodynamic after autonomic blockade and PE doses as well as data between time points: ‘on support’ and 6,000 rpm, 6,000 rpm and 11,000 rpm within groups and 6,000 rpm before and after esmolol were made using a One-Way ANOVA with repeated measures design (SigmaStat 2.03, Systat Software Inc., Point Richmond, Calif.). If the F-ratio was found to exceed a critical value (α<0.05) the post hoc Bonferroni's method was applied to perform pair-wise comparisons. The slopes of the PRSW (M_{W}) and the TP/EDP (M_{TP}) relationships were derived from least squares linear regression analysis of plots (2-second averages) for the SW versus the end-diastolic volume and for the TP versus the LVEDP, respectively. Multiple linear regression analysis (??? Program used w/info) was used to compare M_{TP} and M_{W} before and after esmolol infusion and to compare M_{TP} after autonomic blockade and PE infusion.

Results

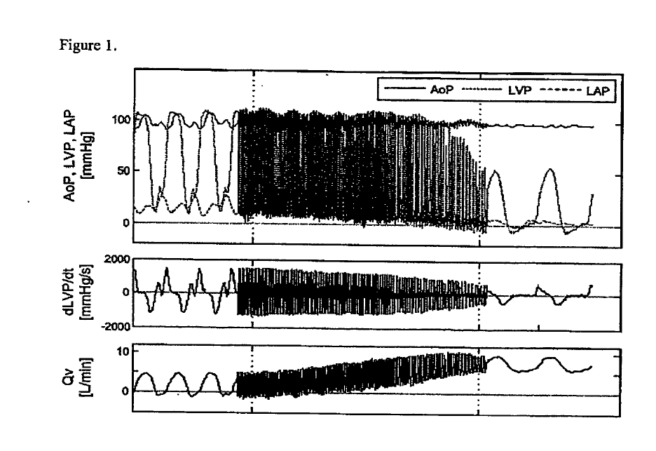

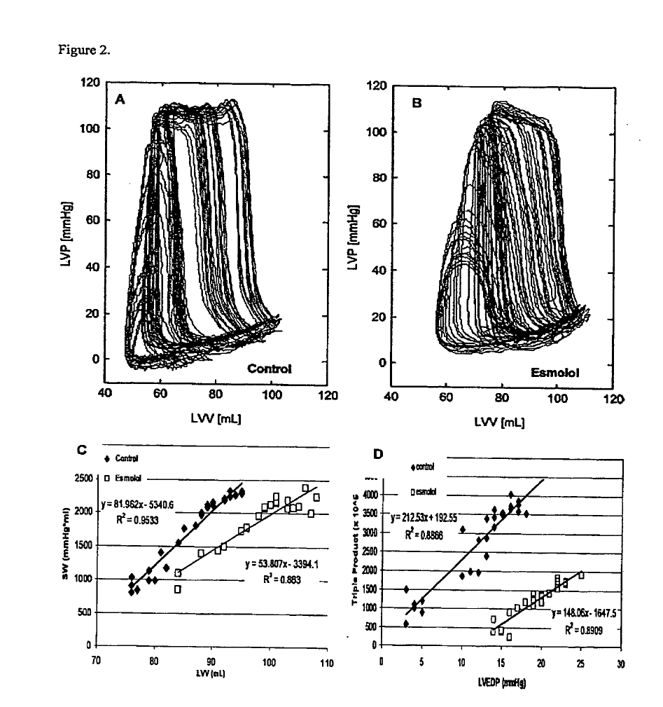

Six animals were studied after LVAD implantation and instrumentation. Animals were partially supported with the LVAD on average for 13 days (range 3 to 40 days). A typical ‘Run’ progressed from 6,000 rpm to 10,880±120 rpm. Representative hemodynamic tracings during LVAD unloading ‘run’ are shown in FIG. 1 with a resulting set of P-V loops before and after esmolol in FIGS. 2A and 2B. The PRSW and TP/EDP relationships before and after esmolol are shown in FIGS. 2C and 2D, respectively. Esmolol reduced Me from 159±23.8 to 71±15.1 mm Hg*s⁻¹*bpm (N=6; P<0.001) and M_{W} from 117±15.8 to 72±9.4 mm Hg (N4; P<0.001). Right ventricular dP/dtₘₐₓ was reduced after esmolol; otherwise, all other RV hemodynamics were not significantly altered by LV reloading and subsequent LVAD unloading ‘run’. Additional hemodynamic data from ‘runs’ before and after esmolol infusion are presented in Table 1.

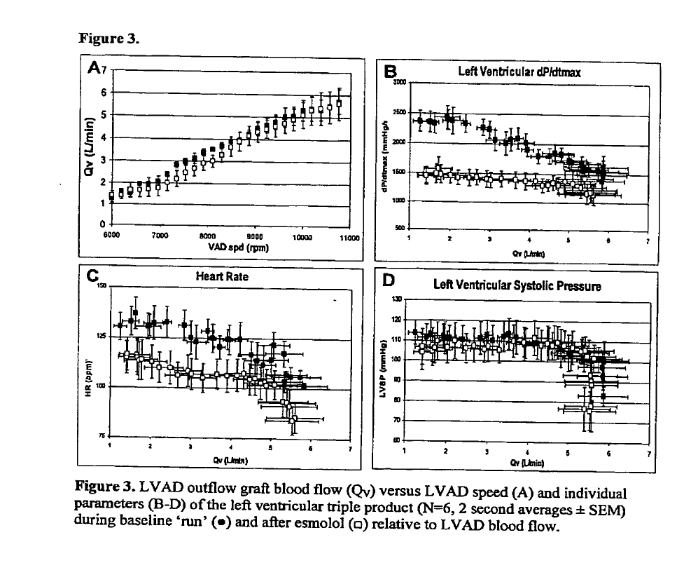

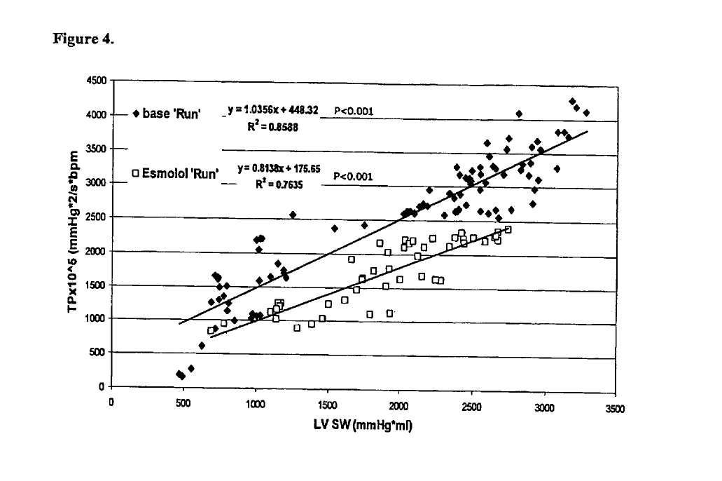

Outflow graft blood flows (Q_{V}) were nearly identical before and after esmolol (FIG. 3A). LVAD blood flow increase linearly with LVAD speed until plateau. Each component of the LV TP (dP/dtₘₐₓ, LVSP, and BR) relative to Q_{V} during ‘run’ is shown in FIGS. 3B, 3C and 3D. The predominant effect of esmolol on the TP/EDP slope was reduced LV contraction velocity (dP/dTₘₐₓ, FIG. 3B) and, although HR was lower after esmolol, the change in HR was not appreciably different after esmolol (FIG. 3C). Left ventricular systolic pressure, in FIG. 3D, was not observed to be significantly reduced until late in the ‘run’. In FIG. 4, linear regression demonstrated a high degree of correlation between TP and SW during LVAD unloading ‘run’ and was expressed by the equation y=1.056x+448.3, R²=0.86; P<0.001 before and by the equation y=0.814x+175.6, R²=0.764; P<0.001 after esmolol.

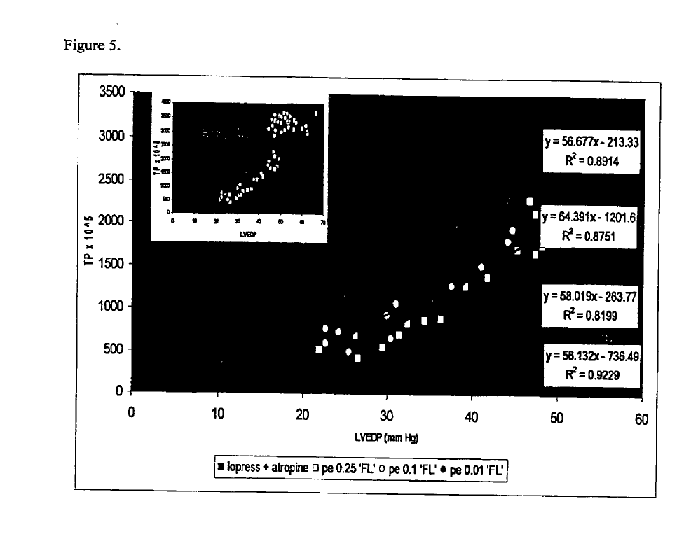

Selected data after autonomic blockade and PE infusion are presented in Table 2. On three separate days in a single animal, infusion of 0.01, 0.1 and 0.25 mcg/kg/min of PE after autonomic blockade increased LVSP by 10.2±2.56, 20.7±1.51 and 27.2±0.93 mm Hg, respectively (P<0.007). Heart rate was 144±4.6 bpm at baseline (pump speed 6,000), 146±5.1 bpm after atropine and 131±1.6 bpm after atropine and metoprolol (P=0.064 vs. baseline; power 0.45, N=3). In FIG. 5, a cluster of points or plateau was noted at higher filling pressures associated with PE infusions (inset), below which, the M_{TP} was observed to be linear. We have defined the position that the M_{TP} resumes a linear relationship as its “flex point” (FIG. 5—inset). There was no appreciable difference in M_{TP} below the ‘flex point’ for each dose of PE (afterload) from that of complete autonomic blockade; even though, M_{TP} was reduced with autonomic blockade (Table 2 and FIG. 5). In this same animal, a plateau [glower et al 1985] was not observed in the TP/EDP relationship either before or after complete autonomic blockade, or on a separate day after esmolol alone (e.g. in FIG. 2). Accounting for PE dose and day, the variability in M_{TP} was 4.59±0.68 (7.0±1.17%) despite the higher variability observed in the TP: 673±92.6 (33.4±4.7%).

Discussion

The ability to quantify systolic myocardial performance is essential for the development of strategies to effectively utilize left ventricular assist devices to ‘Bridge to Recovery’ and also to potentially improve strategies aimed at reducing morbidity for patients ‘Bridged’ to either ‘Transplantation’ or ‘Destination’ therapies. If the currently poor success rate for ‘Bridge to Recovery’ therapy in chronic heart failure patients is a valid metric, then the current methods of functional, metabolic, histological and molecular assessment has, unfortunately, proved to be of little value in improving ‘Bridging’ strategies. Of particular importance, and beyond the mere ability to quantify systolic performance, is the need for frequent and reliable assessments of cardiac function both in the context of directing concomitant therapy and also for the institution of a closed-loop feedback mechanism between sensor and device. As a proof of concept and in light of emerging technology for the assessment of LV pressure in LVAD supported patients (LVP1000®), we compared a potential LV pressure-derived index of cardiac function, TP/EDP, to the PRSW in sheep supported with an axial flow LVAD. Several issues regarding the application of the TP/EDP for the assessment of preload-recruitable LV function are discussed.

Axial Flow LV-Unloading and Pressure-Volume Analysis

The ESPVR (slope: Eₑₛ) as an index of cardiac function is reliant on the coupling of LV end-systolic pressure with end-systolic volume, a correlation directly compromised by axial flow LV unloading. Axial flow supports the systemic circulation and essentially preserves the end-systolic pressures (FIGS. 2A and 2B) until LV volume is insufficient to allow LV ejection (LVSP<MAP, FIG. 1). Thus, a situation is created where the changes in the end-systolic pressure are not dependent on changes in LV volume (i.e. ESPVR), making the Eₑₛ a poor estimate of cardiac function when varying volume with a continuous flow LVAD.

Like the ESPVR, the preload-recruitable stroke work (PRSW), relies on P-V analysis to provide a load-independent index of cardiac function. However, unlike the ESPVR, the slope of the PRSW (M_{W}) remains sensitive to cardiac functional status during axial flow LV-unloading because both LV SW and end-diastolic volume (EDV) vary dependently with the degree of axial flow support. Moreover, the PRSW is linear over a wider range of LV volumes than the ESPVR [Takaoka H, Suga H, Goto Y, Hata K, Takeuchi M. Cardiodynamic conditions for the linearity of the preload recruitable stroke work. Heart and Vessels. 1995; 10(2): 57-68.], a situation we have recently demonstrated for LV unloading with an axial flow LVAD. Unfortunately, the greatest challenge with P-V derived indices of cardiac function is the complexity of repetitively measuring LV volumes in LVAD supported patients.

Left Ventricular Triple Product and the TP/EDP

The LV TP as defined here was intended to provide a surrogate of SW derived from the LV pressure signal by accounting for pressure, heart rate and contractility (i.e. dP/dtₘₐₓ). The dP/dtₘₐₓ or the velocity of LV contraction is traditionally known to be a poor measure of intrinsic cardiac contractility because of its reliance on the LV developed pressure, thereby making it preload and afterload dependent in addition to being heart rate dependent. Several studies have demonstrated that estimates of myocardial work that rely on LV dP/dtₘₐₓ typically correlate poorly with myocardial oxygen consumption (MVO₂). This is a fact we do not disagree with relative to the estimation of MVO₂. However, the observation that changes in the TP and resulting TP/EDP correlate with changes in the SW and resulting PRSW (respectively) is intriguing because it would appear to be inconsistent with others studies. The reasons for the linear correlation between TP and SW may be specific to the method used for LV unloading (i.e. LVAD) in this report.

As demonstrated in FIGS. 1 and 3B, left ventricular dP/dtₘₐₓ was progressively and linearly reduced during LV unloading with an axial flow LVAD, an observation not apparent with vena cava occlusions (data not shown). Vena cava occlusions quickly reduce the LV developed pressure that reduces the LV dP/dtₘₐₓ nonlinearly and, therefore, confounds the interpretation of dP/dtₘₐₓ as a measure of ‘intrinsic contractility’. As previously stated, when reducing LV preload with a continuous flow LVAD, the LV systolic and developed pressures are relatively well preserved (FIGS. 1 and 3D) because the systemic circulation is supported by the LVAD—a very different event from vena cava occlusion. Therefore, changes in dP/dtₘₐₓ are likely more reflective of meaningful changes in preload affecting the contractile state of the myocyte (preload recruitable function) and also possibly minimizing reflex activation (see section: Reflex Activation).

A further consideration of the estimation of cardiac work involves the observation that the HR was progressively reduced with continuous flow LVAD unloading—a phenomenon not yet formally described in the Literature nor completely clarified by these studies. This reduction in HR is unlikely to be mediated by autonomic reflexes as neither atropine or β1 adrenergic blockade altered this progressive bradycardia (FIG. 3C). Furthermore, changes in left atrial pressures (decrease) and loss of pulsatility within the aorta should produce a reflex tachycardia even in light of normal venous pressures and preserved MAP. Though this response is truly intriguing and worthy of further study and characterization, the effect of the decreased HR on TP was in line with known changes in SW and MVO₂ during LVAD unloading. Therefore, and perhaps because of continuous flow LV unloading, changes in HR contribute meaningfully to the observation that the TP correlated well with changes in stroke work and the TP/EDP was linear in almost all cases.

The Linearity of the M_{TP}

The fact that TP was linearly related to changes in LVEDP is an important issue with regards to the utility of the TP/EDP for the assessment cardiac function. The Frank-Starling (F-S) relationship is known to be curvilinear—with a plateau evident at higher filling pressures. Glower and colleagues demonstrated, as Sarnoff and Berglund hypothesized, that substituting EDV for EDP would make the F-S relationship linear. We, however, found the TP/EDP to be linear over the fill range of LV volumes studied with the exception of PE infusion after autonomic blockade, where a plateau was observed at the highest filling pressures. However, the M_{TP} varied little between days and doses of phenylephrine below this plateau—or below the so-called “flex point”. Do the experimental conditions of increased afterload combined with autonomic blockade in this single animal mimic the expected results in experimental or clinical heart failure? Quite possibly, and this is a question we are currently exploring.

Although, the possibility exists that a plateau in the Mob would not routinely be observed in clinical heart failure cases. In all the sheep studied, the LVEDP was elevated upon reloading of the LV prior to a ‘run’ (before esmolol Δ5.0±1.6 mm Hg and after esmolol Δ11.5±1.9 mm Hg), a level of acute volume loading that should theoretically be sufficient to reveal a plateau. However, in all animals studied, no plateau was observed even after β1 blockade (esmolol). Another explanation for the lack of an observed plateau in all studies was that the approximate 1 L/min of flow (@ 6,000 rpm) still present upon LV reloading was sufficient to prevent the observation of a plateau, i.e. prevented full reloading of the left ventricle.

However, we believe based on the few animals studied thus far that another likely explanation for the preserved linearity of the TP/EDP, and again a condition specific to the method of LVAD unloading, was that changes in LVEDP during LVAD unloading were relatively small per unit time in the face of preserved right sided and systemic hemodynamics (Table 1). Acute right ventricular collapse causing septal bulging during vena cava occlusion has been posited as an explanation for the non-linearity of the F-S relationship reliant on LVEDP [Olsen C O. Tyson G S, Maier G W, Spratt J A, Davis J W, Rankin J S. Dynamic ventricular interaction in the conscious dog. Circ Res. 1983; 52: 85, and Glower]—an event that would not be applicable during acute LVAD unloading. Thus, the small incremental changes in LVEDP in the face of supported right ventricular pressures and supported pericardial pressures [Tyson G S. Maier G W, Olsen C O, Davis J W, Rankin J S. Pericardial influences on ventricular filling in the conscious dog: an analysis based on pericardial pressure. Circ Res. 1984; 54: 173.]during a ‘run’ likely allowed the LV TP to linearly reflect changes in LVEDP or to remain better coupled with changes in LV end-diastolic pressure. Furthermore, we believe this [coupling] would hold true for clinical cases of heart failure.

Irregardless of afterload sensitivity or reflex activation (next section), if the TP/EDP was observed to be curvilinear in clinical heart failure cases, then the point where TP assumes a linear relationship to EDP, ‘flex point’, maybe of additional diagnostic and prognostic importance. The ‘flex point’ could hypothetically be a target for support—e.g. 75% of flex. Furthermore, the ‘flex point’ would be data not traditionally available from the P-V relationship and additional study would be needed if plateauing of the M_{PT} proves in the future to be clinically or experimentally evident.

Reflex Activation

Among potential confounding issues related to LV unloading is autonomic reflex activation. Foremost among these would be the impact to the right sided and systemic hemodynamics. The right ventricular hemodynamics were unaltered during LVAD unloading ‘runs’ (Table 1). Therefore, it is unlikely that altered venous filling pressures would have contributed in any substantial way to alter autonomic tone in any particular direction. However, the potential for alteration in arterial and left atrial hemodynamics during unloading ‘run’ still exists.

Left atrial baroreflex activation (Bainbridge reflex) upon reloading of the atria could have affected the TP/EDP relationship. Increased sympathetic drive was evident upon reloading of the LV as both esmolol (N=6 animals) and complete autonomic blockade (N=3 days) reduced, though not significantly (power=0.45), the observed increase in HR (Tables 1 and 2). Additionally, vagal withdrawal, also associated with the Bainbridge reflex, was nearly complete given that atropine administration (N=3 days) did not further increase heart rate above that of LV reloading alone. So it would seem likely that reflex activation and then its subsequent withdrawal—during LVAD unloading—could have affected the TP/EDP. The only evidence contrary to this or that would support a minimal role for the impact of reflex activation is from a comparison we recently reported of the PRSW obtained during an inferior vena cava occlusion and later during a LVAD unloading ‘run’ [McConnell/Sun.jhlt letter]—theoretically, the vena cava occlusion would be completed prior to reflex activation (<10 seconds). No difference was observed in the M_{W} or its intercept based on the method of LV unloading. Unfortunately, it is probably not hemodynamically valid to compare the TP/EDP during a vena cava occlusion with that obtained during a LVAD ‘run’ because, as described earlier, of the reliance of the dP/dtₘₐₓ on the LV developed pressure. This further illustrates that the validity of the TP/EDP would likely not be applicable to all situations of LV unloading—preload should vary independent of afterload for the TP/EDP to be meaningful.

As previously alluded, lowered arterial systolic and pulse pressures, even in the face of maintained MAP, are known to reflexively increase sympathetic drive and mediate vagal withdrawal leading to increase inotropy and heart rate, respectively and concomitantly. However, it is unlikely that the modest changes in aortic systolic pressure would be a primary stimulus to activate aortic baroreceptors, but the loss of aortic pulsatility may have been sufficient to also increase sympathetic efferent tone upon LV reloading (Bainbridge reflex). The question remains what would be the dominant reflex response given an increased pulsatility upon reloading? Therefore, what would be the subsequent effect of the loss of aortic pulsatility during LVAD unloading have on the TP/EDP relationship? Perhaps the competition between afferent and efferent signals accounted for the lack of a difference in the PRSW we observed with an inferior vena cava occlusion (no reflex activation) compared to LVAD unloading. Further studies are warranted to better understand the impact of reflex activation during LVAD unloading of the left ventricle.

Pulsatile versus Non-Pulsatile Support

Debate exists and likely will continue to exist regarding the impact that device type (e.g. pulsatile and non-pulsatile LVADs) will have on supporting patients to ‘Recovery’. Though, pulsatile LVADs more closely recreate the native systemic circulation, the effect of variable loaded beats on the heart due to the often non-synchronous contraction of the LVAD versus the heart is not known. In comparison to pulsatile pumps, axial-flow LVADs are equally effective at reducing LV preload and supporting the systemic circulation. To the possible credit of continuous flow devices, they do unload the LV in a more consistent/continuous manner and, therefore, may be better suited for ‘weaning’ strategies. Clearly, the methods described herein for assessing cardiac function would be reproducible only while a patient was supported with a continuous flow device (i.e. axial or centrifugal). Whether indices such as the TP/EDP will prove to be of sufficient value over simple pressure derived data at better identifying patients for and then accomplishing successful ‘Bridge to Recovery’ strategies and, thereby, conferring an advantage to those patients supported with a continuous flow device is yet another question for the future of these intersecting technologies.

### CONCLUSIONS

The relationship of TP to EDP (TP/EDP), derived solely from the LV pressure signal in sheep partially supported with a continuous flow LVAD (Heartmate II®), establishes a proof of concept methodology for the assessment of LV function in patients supported with an left ventricular assist device. The use of an axial flow LVAD to acutely reload and then unload the LV (‘run’) for the purposes of establishing preload recruitable function was reliable and reproducible. The LV triple product correlated with stroke work during LVAD ‘runs’. Also, changes observed in the TP/EDP relationship (slope: M_{TP}) were similar to those observed in the PRSW and reflect alterations in cardiac inotropy in LVAD supported sheep. Preliminary data demonstrated that the M_{TP} was independent of physiological conditions of increased afterload. Though the method for assessment of TP/EDP as detailed here is likely only amenable to continuous flow LVADs, left ventricular pressure data should prove valuable in all patients supported with mechanical circulatory support—especially in those instances where criteria for and the potential to ‘wean’ are critical: e.g. post-cardiotomy cardiogenic shock, pregnancy-associated cardiogenic shock and acute myocarditis.

Clearly, positing this concept generates more questions than have been completely answered by this initial report. In general, what specific technical and theoretical limitations exist in the application of P-V analysis or its surrogates to the LVAD supported left ventricle? What specific mechanisms account for the correlation of the TP/EDP with true P-V analyses such as the PRSW—e.g. observed linearity of the Mn despite reliance on the LV end-diastolic pressure [Glower 1985, Olson 1983, Tyson 1984]? What is the contribution and potential impact of autonomic reflex activation during LV reloading and subsequent unloading of the left ventricle? What would be the anticipated differences in patients versus the experimental data presented? Therefore, further investigation and validation of methodologies employing LV pressure-derived data are needed experimentally and clinically before the full utility of such data can be translated to better ‘Bridging’ strategies.

TABLE 1
Hemodynamics During LV Unloading ‘Run’ with an Axial-Flow Left
Ventricular Assist Device (Heart Mate II ®) before and after
β1-Adrenergic Blockade.

| | | | | Esmolol | |
|---|---|---|---|---|---|
| | | Baseline | | (5 mg/kg/min) | |
| | On Support | | ˜10,880 | 6,000 | ˜10,880 |
| | ˜9,000 rpm | 6,000 rpm | rpm | rpm | rpm |
| Arterial | | | | | |
| HR | 119 ± 7.1 | 131 ± 9.9 | 77 ± 8.6‡ | 118 ± 7.9 | 66 ± 14.2‡ |
| BPM | | | | | |
| Q_{V} | 4.5 ± 0.31 | 1.2 ± 0.25⁺ | 5.8 ± 0.71‡ | 1.7 ± 0.21§ | 5.5 ± 0.53‡ |
| L/min | | | | | |
| SBP | 106 ± 2.4 | 118 ± 4.1⁺ | 110 ± 4.4 | 105 ± 6.4§ | 106 ± 5.3 |
| mm Hg | | | | | |
| DBP | 89 ± 5.1 | 90 ± 4.4 | 103 ± 4.4* | 85 ± 5.5 | 100 ± 4.7‡ |
| mm Hg | | | | | |
| MAP | 97 ± 4.2 | 103 ± 4.7 | 104 ± 4.6 | 95 ± 5.7 | 101 ± 5.0 |
| mm Hg | | | | | |
| aBP | 14.5 ± 0.98 | 28.3 ± 2.18⁺ | 6.3 ± 1.04‡ | 20.3 ± 2.72§ | 6.36 ± 0.91‡ |
| mm Hg | | | | | |
| Left Ventricular | | | | | |
| LV SP | 108 ± 3.3 | 116 ± 5.2 | 79 ± 8.1‡ | 105 ± 6.4§ | 79 ± 10.4 |
| mm Hg | | | | | |
| LV EDP | 13.2 ± 1.50 | 18.2 ± 1.17⁺ | 9.7 ± 1.75‡ | 24.4 ± 2.18§ | 15.3 ± 1.83‡ |
| mm Hg | | | | | |
| LV dP/dTₘₐₓ | 2182 ± 231 | 2286 ± 236 | 1431 ± 282* | 1567 ± 194§ | 1184 ± 207 |
| mm Hg*s⁻¹ | | | | | |
| LV dP/dTₘᵢₙ | −2053 ± 128 | −2219 ± 79 | −1155 ± 182* | −1688 ± 147 | 1081 ± 173 |
| mm Hg*s⁻¹ | | | | | |
| Tau | 30.6 ± 2.22 | 34.0 ± 2.87 | 19.3 ± 2.39‡ | 46.2 ± 5.38§ | 40.3 ± 6.71 |
| TP | 2970 ± 299 | 3127 ± 397 | 1019 ± 335‡ | 1847 ± 314§ | 947 ± 245 |
| mmHg⁻¹*s⁻¹* bpm | | | | | |
| SW (N = 4) | 2216 ± 423 | 2455 ± 451 | 1302 ± 189‡ | 1646 ± 388§ | 1093 ± 344 |
| mm Hg^{*}mL | | | | | |
| Right Ventricular | | | | | |
| (N = 5) | | | | | |
| RV SP | 29.1 ± 3.10 | 32.4 ± 4.43 | 29.4 ± 4.00 | 33.6 ± 3.89 | 29.2 ± 3.23 |
| mm Hg | | | | | |
| RV mDP | 4.63 ± 2.08 | 5.70 ± 2.46 | 4.73 ± 2.44 | 8.04 ± 1.98 | 6.63 ± 2.25 |
| mm Hg | | | | | |
| RV dP/dTₘₐₓ | 1084 ± 213 | 1123 ± 225 | 895 ± 92 | 75O ± 121§ | 763 ± 115 |
| mm Hg*s⁻¹ | | | | | |
| LV dP/dTₘᵢₙ | −733 ± 89 | −829 ± 139 | −728 ± 88 | −702 ± 81 | −561 ± 40 |
| mm Hg*s⁻¹ | | | | | |

All data mean ± sem, N = 6.
Comparison (ANOVA RM)
*P < 0.05 from 6,000 rpm w/in groups,
⁺P <0.05 from ‘on support’ to 6,000 rpm,
‡P < 0.01 from 6,000 rpm w/in groups and
§P < 0.05 between groups at 6,000 rpm.
LV: left ventricle,
RV: right ventricle,
HR: heart rate,
Q_{V}: assist device blood flow,
SBP: systolic blood pressure,
DBP: diastolic blood pressure,
MAP: mean arterial pressure,
aBP: aortic beat pressure,
SP: systolic pressure,
EDP: end-diastolic pressure,
mDP: mean diastolic pressure,
Tau: time constant of LV relaxation (Weiss method),
TP: triple product × 10⁵,
SW: LV stroke work.

TABLE 2
Responses after autonomic blockade* to phenylephrine (PE) infusion
in a single animal.

| | LV SP | LV EDP | dP/dtₘₐₓ | HR | TP | M_{TP} | P_{TP} |
|---|---|---|---|---|---|---|---|
| | mm Hg | mm Hg | mm Hg/s | Bpm | mm Hg²*s⁻¹*bpm | mm Hg²*s⁻¹*bpm | (M_{TP} x-intercept) |
| | (P-value)⁺ | (P-value)⁺ | (P-value)⁺ | (P-value)⁺ | (P-value)⁺ | (P-value)⁺ | (P-value)⁺ |
| Baseline | 114.4 ± 4.6 | 36.4 ± 2.6 | 2078 ± 150 | 144 ± 4.6 | 3242 ± 364 | 120.2 ± 16.3 | 6.9 ± 3.1 |
| | (0.088) | (1.00) | (0.012) | (0.064) | (0.025) | (0.05) | (1.00) |
| Autonomic | 107.8 ± 5.2 | 42.5 ± 2.9 | 1530 ± 142 | 131 ± 1.6 | 2085 ± 293 | 69.0 ± 7.3 | 5.4 ± 3.4 |
| blockade | | | | | | | |
| PE 0.01† | 118.0 ± 2.7 | 43.6 ± 5.5 | 1801 ± 230 | 135 ± 1.5 | 2705 ± 419 | 72.4 ± 7.8 | 13.9 ± 4.8 |
| | (0.007) | (1.00) | (0.415) | (1.00) | (0.492) | (1.00) | (1.00) |
| PE 0.10† | 128.5 ± 5.5 | 50.0 ± 3.5 | 1904 ± 170 | 136 ± 0.5 | 3143 ± 398 | 71.8 ± 9.4 | 13.7 ± 1.8 |
| | (<0.001) | (0.780) | (0.103) | (0.343) | (0.042) | (1.00) | (1.00) |
| PE 0.25† | 135.0 ± 4.6 | 56.3 ± 3.9 | 1899 ± 180 | 137 ± 1.9 | 3301 ± 380 | 75.0 ± 5.6 | 12.3 ± 6.1 |
| | (<0.001) | (0.059) | (0.109) | (0.391) | (0.019) | (1.00) | (1.00) |

Data is mean ± SEM with pump at 6,000 rpm (N = 3 days).
*atropine (0.1 mg/Kg) and metoprolol (5 mg).
⁺ANOVA RM versus autonomic blockade.
†mcg/Kg/min.
LV SP: left ventricular systolic pressure,
EDP: end-distolic pressure,
dP/dtmax: maximum derivative of pressure versus time,
HR: heart rate,
TP: triple product,
M_{TP}: slope of TP/EDP relationship,
P_{TP}: pressure at zero TP (x-intercept).

1. A device for performing the method substantially as described herein.
A method for determination of cardiac function by monitoring left ventricular (LV) pressure and varying ventricular assist device (VAD) speed, utilizes a relationship of the end-diastolic LV pressure (LVEDP) to an estimate of LV work calculated from the LV pressure signal by the triple product (TP): dp/dtmax*HR*LVSP, wherein the slope of a regression analysis of the comparison of TP vs LVEDP is indicative of a patient's cardiac efficiency and analogous and comparable to preload recruitable stroke work as calculated from direct volume measurement of the LV, and of native cardiac function of a patient supported by a VAD.
